(19) United States
(12) Patent Application Publication
(10) Pub. No.: US 2011/0028801 A1
(43) Pub. Date: Feb. 3, 2011

(54) CARDIOVASCULAR VESSEL ELASTICITY MONITORING

(75) Inventor: Steve Koh, South Pasadena, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/511,484

(22) Filed: Jul. 29, 2009

(51) Int. Cl. A61B 5/0205; A61B 5/02

(52) U.S. Cl. 600/301; 600/500; 600/481

(57) ABSTRACT

Elasticity of a cardiovascular vessel of a patient is monitored to provide an indication of whether the patient's health is changing. In some aspects the elasticity of a cardiovascular vessel is determined irrespective of the current blood pressure level of the patient at the time the elasticity is determined. For example, vessel elasticity may be determined based on a defined set of data that maps vessel elasticity with reflectance times for different blood pressure levels. In some implementations, this set of data corresponds to a set of iso-pressure lines.

### TECHNICAL FIELD

This application relates generally to medical monitoring devices and methods and more specifically, but not exclusively, to monitoring elasticity of a cardiovascular vessel.

### BACKGROUND

A change in the elasticity of a cardiovascular vessel of a patient may serve as an indicator of the cardiac health of the patient. For example, in the event blood flow to an organ decreases due to worsening cardiac function, the body (e.g., via a hormonal mechanism) may automatically compensate for this reduction in blood flow by increasing blood pressure. If the blood pressure remains high for a prolonged period of time, vessel walls of the cardiovascular system may thicken in an attempt to “hold back” the increased blood pressure. This thickening of a vessel wall may, in turn, reduce the elasticity of the vessel wall. Thus, a decrease in the elasticity of a cardiovascular vessel may indicate that the cardiac health of the patient is worsening. Accordingly, there is a need for effective techniques for determining the elasticity of cardiovascular vessels of a patient to provide improved health monitoring for the patient.

### SUMMARY

A summary of several sample aspects of the disclosure and embodiments of an apparatus constructed or a method practiced according to the teaching herein follows. It should be appreciated that this summary is provided for the convenience of the reader and does not wholly define the breadth of the disclosure. For convenience, one or more aspects or embodiments of the disclosure may be referred to herein simply as “some aspects” or “some embodiments.”

The disclosure relates in some aspects to monitoring the elasticity of a cardiovascular vessel of a patient. Here, changes in the elasticity of a cardiovascular vessel may serve to indicate whether the patient's cardiac health is worsening (e.g., due to a decrease in cardiac function) or improving (e.g., due to current therapy). For example, a patient's blood pressure may drop at the onset of heart failure. To compensate for this drop in blood pressure, the patient's body may release hormones (e.g., catecholamine) to stiffen the vessel walls (e.g., arterioles, arteries, etc.) of the cardiovascular system. Accordingly, the monitoring techniques described herein may be used to detect changes in peripheral resistance caused by neurohormonal insult. Thus, the results of the monitoring may be used to determine whether to prescribe (e.g., adapt) therapy for the patient.

The disclosure relates in some aspects to determining the elasticity of a cardiovascular vessel irrespective of the current blood pressure level of the patient at the time the elasticity is determined. For example, elasticity may be determined based on the reflectance time of a pressure wave in the vessel, whereby a longer reflectance time indicates higher vessel elasticity. Here, the reflectance time may be defined as the time between a specified time (e.g., the time of peak amplitude) associated with the pressure waveform and a specified time associated with a reflection of the pressure wave off of a vessel wall.

In practice, the reflectance time also may depend on the current blood pressure. Specifically, a higher blood pressure may result in a shorter reflectance time. Thus, reflectance time measurements made at different blood pressure levels may not accurately indicate whether there has been a change in the elasticity of the vessel or the degree of such a change.

To mitigate this problem, a monitoring scheme as taught herein may employ a defined set of data that maps vessel elasticity with reflectance times for different blood pressure levels. In some implementations, this set of data corresponds to a set of iso-pressure lines each of which maps reflectance time to elasticity for a given blood pressure level. Accordingly, the current blood pressure level of a patient may be measured in conjunction with measuring reflectance time. This information and the data set may then be used to provide an accurate (e.g., blood pressure independent) elasticity estimate.

### BRIEF DESCRIPTION OF THE DRAWINGS

These and other features, aspects, and advantages will be more fully understood when considered with respect to the following detailed description, the appended claims, and the accompanying drawings, wherein:

FIG. 1 is a simplified block diagram of an embodiment of an apparatus configured to provide functionality for determining cardiovascular vessel elasticity;

FIG. 2 is a simplified flowchart of an embodiment of operations that may be performed in conjunction with determining cardiovascular vessel elasticity;

FIG. 3 is a simplified diagram illustrating sample reflectance times of pressure waves in a cardiovascular vessel;

FIG. 4 is a simplified diagram illustrating sample iso-pressure lines;

FIG. 5 is a simplified flowchart of an embodiment of operations that may be performed in conjunction with monitoring cardiovascular vessel elasticity and adapting therapy based on the monitoring;

FIG. 6 is a simplified diagram of an embodiment of a monitoring system including an implantable medical device and an external monitor device;

FIG. 7 is a simplified diagram of an embodiment of an implantable stimulation device in electrical communication with one or more leads implanted in a patient's heart for sensing conditions in the patient, delivering therapy to the patient, or providing some combination thereof; and

FIG. 8 is a simplified functional block diagram of an embodiment of an implantable cardiac device, illustrating basic elements that may be configured to sense conditions in the patient, deliver therapy to the patient, or provide some combination thereof.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

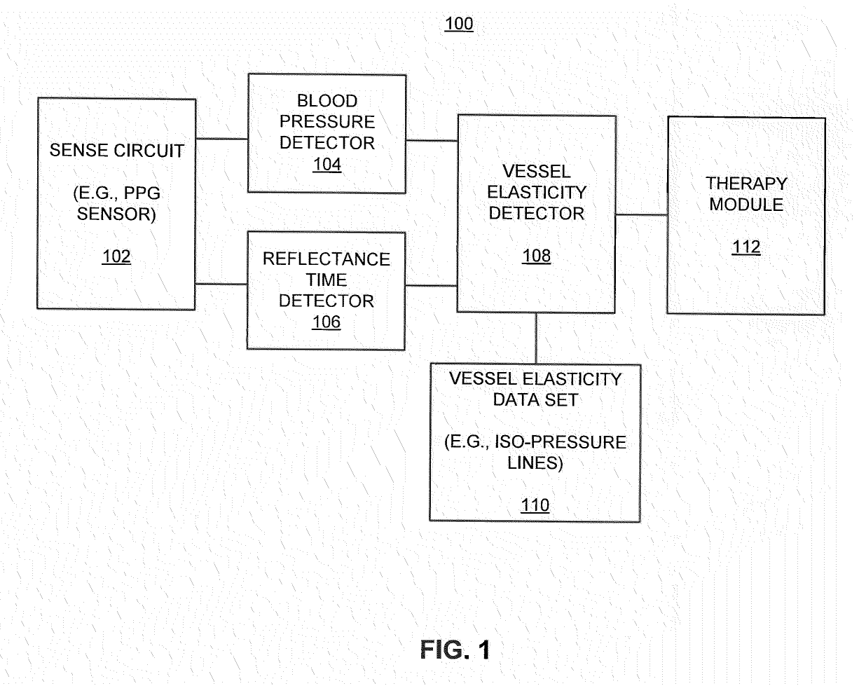

FIG. 1 illustrates components of an apparatus 100 (e.g., an implantable medical device) that provide functionality relating to monitoring the elasticity of a vessel of the cardiovascular system of a patient. Briefly, one or more sense circuits (referred to herein for convenience as sense circuit 102) that may be implanted in the patient sense physiologic conditions and generate signals that are used by a blood pressure detector 104 to determine the patient's blood pressure and by a reflectance time detector 106 to determine reflectance times of pressure waves traveling within the cardiovascular system. A vessel elasticity detector 108 uses the determined blood pressure and reflectance time information, along with information from a vessel elasticity data set 110 to determine the elasticity of the cardiovascular vessel (e.g., determine whether there is a trend of increasing or decreasing elasticity). A therapy module 112 may then identify a condition indicative of the patient's cardiac health based on any changes in the vessel elasticity. For example, a change in elasticity may serve as a predictor of heart failure, a hypertension precursor, an indication of medical regimen compliance, an indication of salt intake compliance, an indication of neurohormonal insult leading to wall stiffness (e.g., catecholamine release), or an indication of some other condition of the patient. Thus, a detected change in vessel elasticity may lead to appropriate therapy being prescribed for the patient (e.g., the current therapy may be adapted).

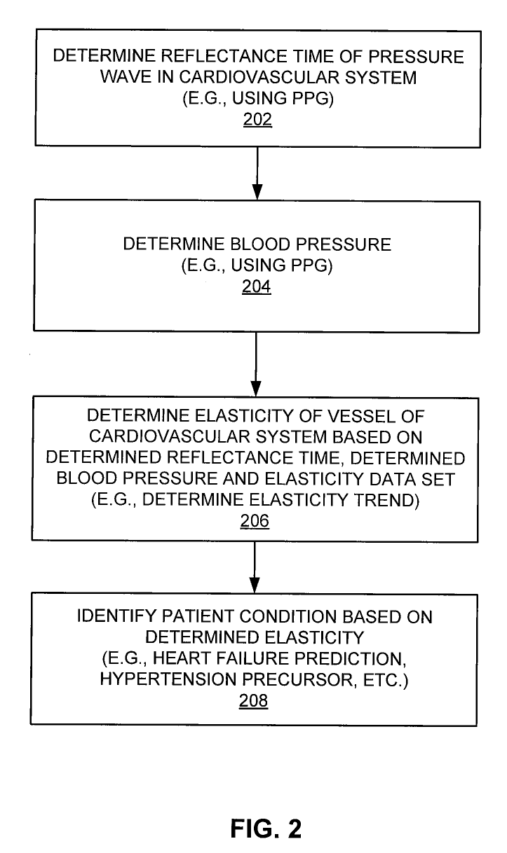

Sample elasticity monitoring operations will be described in more detail in conjunction with the flowchart of FIG. 2. For convenience, the operations of FIG. 2 (or any other operations discussed or taught herein) may be described as being performed by specific components (e.g., the components of FIG. 1). It should be appreciated, however, that these operations may be performed by other types of components and may be performed using a different number of components. It also should be appreciated that one or more of the operations described herein may not be employed in a given implementation.

In this example, vessel elasticity is determined by measuring the reflectance time of a pressure wave. Accordingly, as represented by block 202, the method of FIG. 2 involves determining reflectance time of a pressure wave in the patient's cardiovascular system.

In some implementations reflectance time indicates the time it takes for a pressure wave (e.g., the leading edge of a pressure wave) to travel from a given point in the cardiovascular system, reflect off a vessel wall, and return to that point. It should be appreciated that reflectance time may be defined in others ways (e.g., using different timing reference points) in other implementations.

In general, the elasticity of the vessel wall will effect the reflectance time. For example, a vessel wall with low elasticity will tend to reflect most of the energy from an inbound pressure wave. Consequently, a pressure wave will quickly bounce off such a vessel wall. In contrast, a vessel wall with high elasticity will tend to absorb more of the energy of an inbound pressure wave (i.e., the vessel wall will tend to distort when the pressure wave hits). Thus, a pressure wave will be reflected less quickly off a highly elastic vessel wall.

Reflectance time also may depend on blood pressure. Specifically, higher blood pressure may cause the pressure waves to travel at a higher velocity. As a result, the reflectance time of a pressure wave may be shorter when blood pressure is higher.

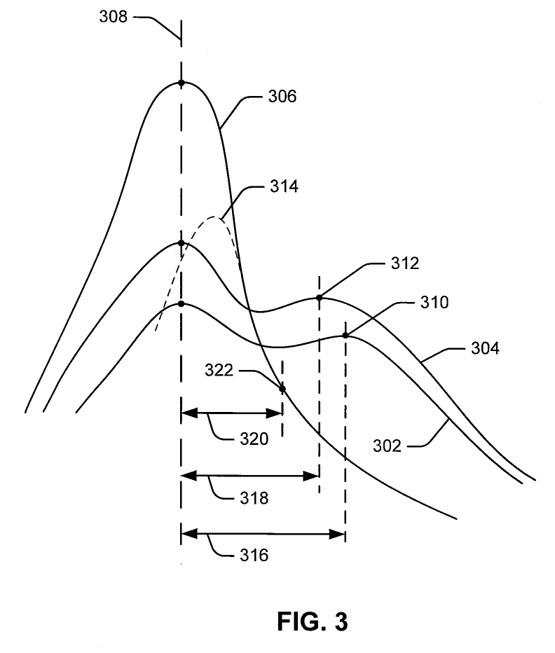

FIG. 3 illustrates, in a simplified manner, sample pressure waveforms 302, 304, and 306. Here, the waveform 302 corresponds to a lower blood pressure, the waveform 304 corresponds to a medium blood pressure, and the waveform 306 corresponds to a higher blood pressure.

The shapes of the waveforms 302, 304, and 306 illustrate how reflectance time may be shorter when blood pressure is higher. Here, it may be seen that at higher pressures, reflected pressure waves occur earlier in the waveforms (more to the left in FIG. 3) since they have faster propagation times. Specifically, at the lower and medium pressures, the reflected pressure waves (with peaks at points 310 and 312) result in dicrotic notches on the trailing edges of the waveforms 302 and 304. Conversely, at higher blood pressures the trailing edges are smoother since the reflected pressure wave (indicated by dashed line 314) merges with the initial peak of the waveform 306.

In the example of FIG. 3, the reflectance times are defined as the time between the peak amplitude (corresponding to dashed line 308) of the waveform and an inflection point (e.g., which may be identified by a derivative of the waveform) on the trailing edge of the waveform. Specifically, a reflectance time 316 for waveform 302 is defined as the time between line 308 and the inflection point 310. A reflectance time 318 for waveform 304 is defined as the time between line 308 and the inflection point 312. A reflectance time 320 for waveform 306 is defined as the time between line 308 and an inflection point 322.

In the example of FIG. 1, the reflectance time of a pressure wave is determined by operation of the sense circuit 102 and the reflectance time detector 106. The reflectance time detector 106 may comprise signal processing functionality that processes a pressure waveform signal received from the sense circuit 102. In a typical implementation, this may involve converting the received signal to a digital signal and performing digital signal processing on the resultant digital signal to determine the reflectance time.

In some implementations the sense circuit 102 comprises a photoplethysmography (“PPG”) sensor that generates a signal indicative of a pressure wave. For example, a PPG sensor may generate a signal representative of a series of waveforms each of which may be similar to a waveform as shown in FIG. 2. Here, the shape of the waveforms may depend, at least in part, on the current blood pressure and the current elasticity of the vessel as discussed above.

A PPG sensor may generate such a signal by measuring changes in blood density. That is, the PPG sensor may generate a signal where the amplitude of the signal changes over time based on changes in the density of hemoglobin in the blood at a given location in a vessel. For example, there may be a temporary increase in the blood density as a pressure wavefront passes through the portion of the vessel monitored by the PPG sensor. Thus, the PPG signal may represent the same information as a conventional blood pressure profile (e.g., acquired by other means).

Other types of sense circuits may be used in other implementations. For example, some implementations may use a lead-based pressure sensor (e.g., located in the arterial system), cardiogenic impedance sensing, or piezo-based sensing to acquire pressure wave information to determine reflectance time.

The cardiovascular vessel (and corresponding vessel wall) referred to above may comprise one or more parts of a cardiovascular system depending on where the sense circuit is (or sense circuits are) positioned. For example, in some cases a sense circuit may be positioned to detect a reflectance time for a pressure wave in an arteriole portion of a cardiovascular system. Thus, the vessel wall in this case may consist of the walls of a set of arteriole branches that are in the vicinity of the measurement point and/or downstream from the measurement point.

In some cases a sense circuit may be positioned to detect a reflectance time for a pressure wave in an artery (e.g., the aorta) of a cardiovascular system. Thus, the vessel wall in this case may consist of the portion of the artery wall that is in the vicinity of the measurement point and/or downstream from the measurement point.

In some cases a sense circuit may be positioned in or near the heart to detect a reflectance time in a portion of the cardiovascular system that is first affected by a contraction-induced pressure wave. Thus, the vessel wall in this case may consist of the portion of one or more cardiac walls and/or arterial walls (e.g., in the aortic arch) that is in the vicinity of the measurement point and/or downstream from the measurement point.

In some cases a sense circuit may be positioned in or near the heart to detect a reflectance time in a portion of the cardiovascular system that is first affected by a contraction-induced pressure wave. Thus, the vessel wall in this case may consist of the portion of one or more cardiac walls and/or arterial walls (e.g., in the aortic arch) that is in the vicinity of the measurement point and/or downstream from the measurement point.

In some cases multiple sense circuits may be employed. Thus, the above-referenced vessel may comprise one or more: arteries, arteriole branches, cardiac walls, or other sections of a cardiovascular system.

A sense circuit may be implanted in various ways. In some cases a sense circuit may be incorporated into an implantable medical device. For example, a sealable opening may be provided in a housing of an implantable device for a sensor. Such a sensor may thus sense physiologic conditions that are indicative of pressure waves in the cardiovascular system at the implant site. As a specific example, this sensor may comprise a PPG sensor that detects changes in blood volume over time (i.e., blood flow) in nearby arteriole branches (e.g., arteriole branches in the chest muscles in the case of a pectoral implant site).

In some cases a sense circuit may be incorporated into an implantable lead. For example, a sense circuit may be incorporated into a distal portion of an implantable lead that is coupled at a proximal end to an implantable medical device. In this way, the distal portion of the lead may be routed to a preferred monitor site while the implantable medical device (e.g., including signal processing and communication circuitry) is implanted at another site (e.g., a site that is more optimal for implant of the larger medical device). In some cases, the lead may include components that provide other functionality. For example, the sense circuit may be incorporated into a cardiac pacing and/or sensing lead that includes electrodes for generating and/or sensing electrical signals. In a typical case, a distal end of such a lead may be implanted in the heart whereby the pressure wave sense circuit may be implanted further up the lead (i.e., away from the distal end) for measuring cardiac wall and/or artery elasticity.

In some cases a sense circuit may be implanted at a subcutaneous location. For example, the sense circuit may be incorporated into a lead or device (e.g., wireless sensing device) that is implanted just under the patient's skin.

Referring again to FIG. 2, to account for the effects the current blood pressure may have on the reflectance determination of block 202, the current blood pressure is determined as represented by block 204. In the example of FIG. 1, the blood pressure is determined by operation of the sense circuit 102 and the blood pressure detector 104. The blood pressure detector 104 may comprise signal processing functionality that processes a pressure waveform signal received from the sense circuit 102. In a typical implementation, this may involve converting the received signal to a digital signal and performing digital signal processing on the resultant digital signal to determine the blood pressure.

Advantageously, in some implementations common circuitry may be used to determine blood pressure and reflectance times. For example, common sense circuitry may be used to acquire signals for blood pressure and reflectance time measurements.

As a specific example, a signal generated by a PPG sensor that is used to determine reflectance time also may be used to determine blood pressure. As mentioned above, a PPG sensor may measure the blood volume over time (i.e., blood flow) in the portion of the cardiovascular system being sensed by the sensor (e.g., the portion of the arterial system that lies underneath the PPG sensor). In a closed system such as a cardiovascular system, the velocity of a pressure wave is proportional to the pressure. Consequently, blood pressure (e.g., relative blood pressure) may be determined by measuring the time it takes for a pressure wave to propagate a certain distance (e.g., a pulse transmission time).

In some implementations this transmission time (e.g., a relative propagation time) is determined by measuring the time between a cardiac event such as an R-wave and a pressure pulse event. For example, a transmission time to be monitored for determining blood pressure may comprise the time from a peak of an R-wave to a foot or peak of a resulting pressure pulse. The current blood pressure may then be estimated based on the measured transmission time.

Other types of sense circuits may be used in other implementations. For example, some implementations may use a piezo-based blood pressure sensor. Also, a blood pressure sensor may be implanted in the heart (e.g., in the right ventricle), in an artery, near the cardiovascular system, or at some other suitable location. In some cases a blood pressure sensor may be incorporated into an implantable lead.

The blood pressure determined (e.g., estimated) at block 204 may take various forms. In some cases peak-to-peak blood pressure may be calculated. In some cases mean blood pressure may be calculated. In come cases relative blood pressure (e.g., the change in pressure from one measurement to the next) may be calculated.

As represented by block 206 of FIG. 2, the elasticity of the vessel may be determined based on the determined reflectance time and blood pressure, along with a data set that that correlates different blood pressure values and different pressure wave reflectance time values to different vessel elasticity values. In some implementations such a data set may comprise a set of iso-pressure lines that map pressure wave reflectance times to vessel elasticity. That is, the data set may include information (e.g., indexed data, functions, or some other form of information) that describes these iso-pressure lines.

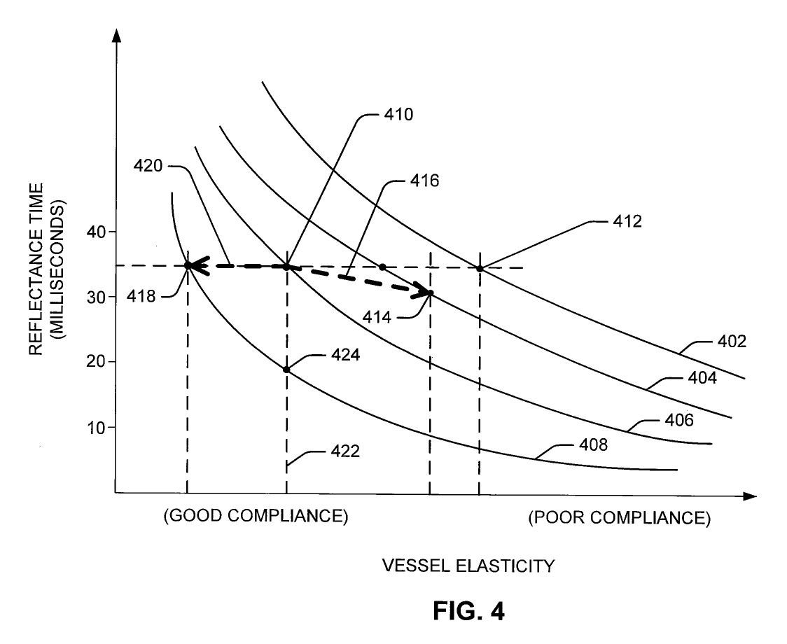

FIG. 4 depicts, in a simplified manner, sample iso-pressure lines 402, 404, 406, and 408. For example, line 402 may correspond to a blood pressure of 80 mm Hg, line 404 may correspond to a blood pressure of 100 mm Hg, line 406 may correspond to a blood pressure of 150 mm Hg, and line 408 may correspond to a blood pressure of 200 mm Hg. Here, each line correlates different reflectance time values with different vessel elasticity values.

The elasticity values correlate to high vessel wall elasticity on the left portion of the x-axis to lower vessel wall elasticity on the right portion of the x-axis. As discussed above, high elasticity may correspond to a healthier patient while low elasticity may correspond to an unhealthy patient. These elasticity ranges may thus be referred to as corresponding to good vessel wall compliance or poor vessel wall compliance as indicated in FIG. 4.

FIG. 4 illustrates that a given value of reflectance time may correspond to good compliance at one blood pressure reading and poor compliance at another blood pressure reading. For example, a reflectance time of 35 ms may correspond to good compliance at point 410 (150 mm Hg) and poor compliance at point 412 (80 mm Hg).

The iso-pressure line information of FIG. 4 may be used to accurately determine the vessel elasticity when different values of reflectance time are obtained at different blood pressure readings. For example, based on the iso-pressure line information, it may be determined that the operating point 410 (at 150 mm Hg) corresponds to the same compliance as the operating point 424 (at 200 mm Hg).

Moreover, trends in the vessel elasticity may be determined based on prior and current measurements. For example, worsening compliance is indicated by a transition from point 410 to a point 414 as indicated by dashed arrow 416. Conversely, improving compliance is indicated by a transition from point 410 to a point 418 as indicated by dashed arrow 420.

In the example of FIG. 1, the vessel elasticity is determined by a vessel elasticity detector 108 that uses a vessel elasticity data set 110 (e.g., stored in a data memory). For example, the vessel elasticity detector 108 may use the blood pressure and reflectance time information received from the blood pressure detector 104 and the reflectance time detector 106, respectively, to identify an operating point along an iso-pressure line defined by the data set 110. In this way, the vessel elasticity detector 108 may determine the current vessel elasticity value and identify any trends in the elasticity of the vessel wall (e.g., increasing or decreasing elasticity).

As represented by block 208 of FIG. 2, one or more patient conditions may then be identified based on the determined elasticity (e.g., based on a determined trend). For example, an indication of a decrease in elasticity may be used to predict the possibility of heart failure or may serve as a precursor of hypertension. In some aspects, medical regimen compliance may be based on a detected a change in elasticity. As an example, if compliance (i.e., elasticity) is worsening, beta blockers may be prescribed for the patient or the dosage may be increased. Similarly, hypertension therapy may be commenced or adapted if compliance is worsening. For salt intake compliance, salt intake may be reduced if compliance is worsening. Also, in some cases vasodilator-based techniques may be employed to dilate a cardiovascular vessel.

In the example of FIG. 1, the apparatus 100 may include a therapy module 112 that uses the vessel elasticity (e.g., trend) information to generate an appropriate indication of the patient's condition. As discussed in more detail below, in some cases the therapy module 112 may simply send an indication of the elasticity or trend to another entity (e.g., an external monitor), whereupon the other entity may process the information or enable a treating physician to view the information so that appropriate therapy may be prescribed, as appropriate. In addition, in some cases the therapy module 112 may cause therapy to be adapted. For example, when the apparatus 100 comprises an implantable cardiac stimulation device, the therapy module 112 may control pacing therapy for the patient. Similarly, when the apparatus 100 comprises an implantable device that includes a drug pump, the therapy module 112 may control the administration of drugs by the drug pump.

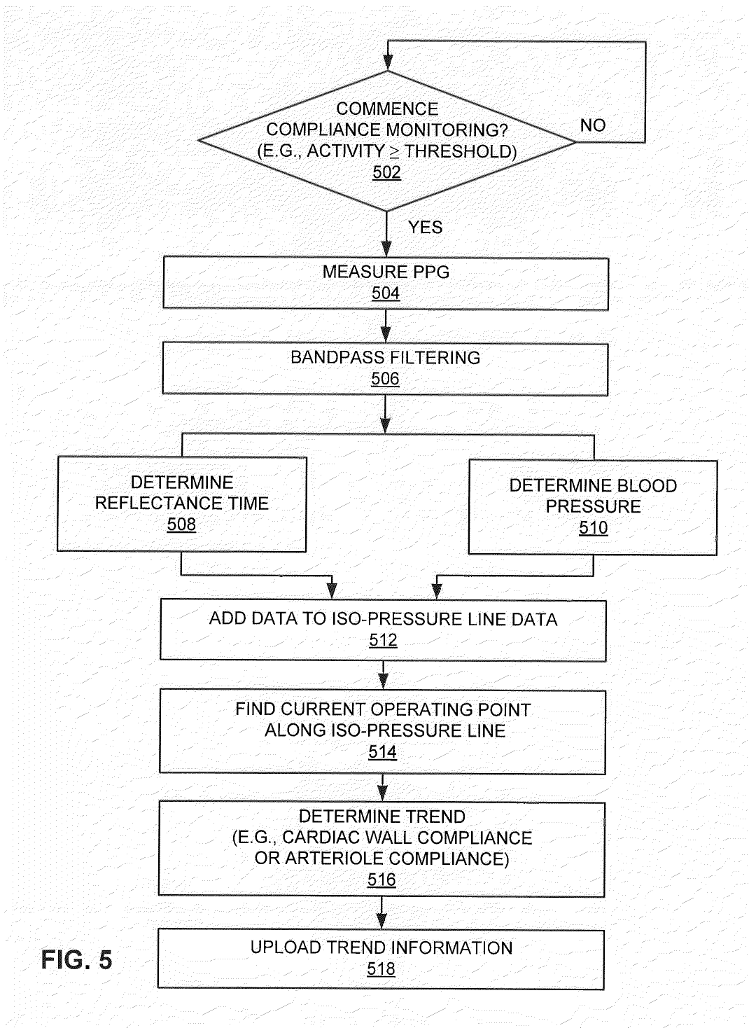

Compliance monitoring as taught herein may be implemented in various ways in different implementations. For illustration purposes, FIG. 5 provides an example of several operations that may be performed in some implementations. In this example, reflectance time and blood pressure are both determined based on PPG measurements.

As represented by block 502, compliance monitoring may be triggered based on one or more conditions. For example, in some cases compliance monitoring may be performed if a patient's activity level is greater than or equal to a threshold activity level.

As represented by block 504, once compliance monitoring is enabled, PPG measurement operations may commence. This may involve, for example, activating the PPG sensing circuitry (e.g., in cases where the sensing is not otherwise enabled) and/or activating signal processing operations that process the raw PPG signals. As mentioned above, PPG techniques may be used to measure cardiac wall compliance, arteriole compliance, or some other form of compliance.

As represented by block 506, in some implementations the PPG signal information may be filtered (e.g., bandpass filtered). For example, a PPG signal may include a respiratory component. Accordingly, filtering may be employed to remove this respiratory component. For example, pressure waves may be sampled over several beats and the resulting signal averaged in an attempt to reduce any respiratory component in the signal.

As represented by block 508, the reflectance time may then be determined based on the PPG signal. For example, as discussed herein, the time between certain events in one or more pressure waveforms may be calculated.

As represented by block 510, the current blood pressure also may be determined based on the PPG signal. As discussed herein, the blood pressure may be estimated based on the calculated velocity of a pressure wave.

As represented by block 512, in some implementations the elasticity data set (e.g., iso-pressure line information) may be based, at least in part, on patient specific information. For example, blood pressure and reflectance time data may be collected over a period of time to establish a baseline for the vessel elasticity. Any deviation from this baseline as detected by subsequent measurements may then serve as an indication that the elasticity has changed. If desired, this information also may take the patient's currently prescribed therapy (e.g., prescribed medications) into account. For example, different baselines may be established for different therapy regimens.

In some implementations the elasticity data set may be downloaded into the monitoring device via an external programmer. For example, the data set may be defined (at least initially) based on empirical information collected from other patients. In some cases, different data sets may be defined for different classifications (e.g., cardiac health classifications, body fat indices, height, weight, medical conditions, and so on).

As mentioned above, the elasticity data set may comprise absolute values or relative values. For example, in cases where a monitoring device estimates relative blood pressure values, the iso-pressure lines of the data set also may correspond to relative blood pressure values. Conversely, in cases where a monitoring device estimates absolute blood pressure values, the iso-pressure lines of the data set also may correspond to absolute blood pressure values.

As represented by block 514, the current operating point within the iso-pressure line data may then be determined based on the reflectance time and the blood pressure determined at blocks 508 and 510, respectively. For example, referring to FIG. 4, if the reflectance time is 35 ms and the blood pressure is 150 mm Hg, a vessel elasticity value corresponding to the vertical dashed line 422 is identified.

As represented by block 516, the current elasticity value may be compared with one or more previously determined elasticity values to identify a trend in the data, if applicable. Thus, it may be determined, for example, whether cardiac wall compliance or arteriole compliance is improving or worsening.

As represented by block 518, in some cases a compliance monitoring report may be uploaded to another entity. For example, an implanted device may generate an indication (e.g., comprising the raw trend data) of a detected compliance trend. This indication may then be transmitted from the implanted device to an external device (e.g., a bedside monitor, an external programmer, or other device). For example, the external device may interrogate the implantable device at regular intervals or information may be uploaded based on some trigger event. The external device may further process the information and display the information or send the information to another device (e.g., a web server) from which the information may be accessed. In this way, the information may be accessed by a physician or other clinician that may prescribe the appropriate therapy for the patient.

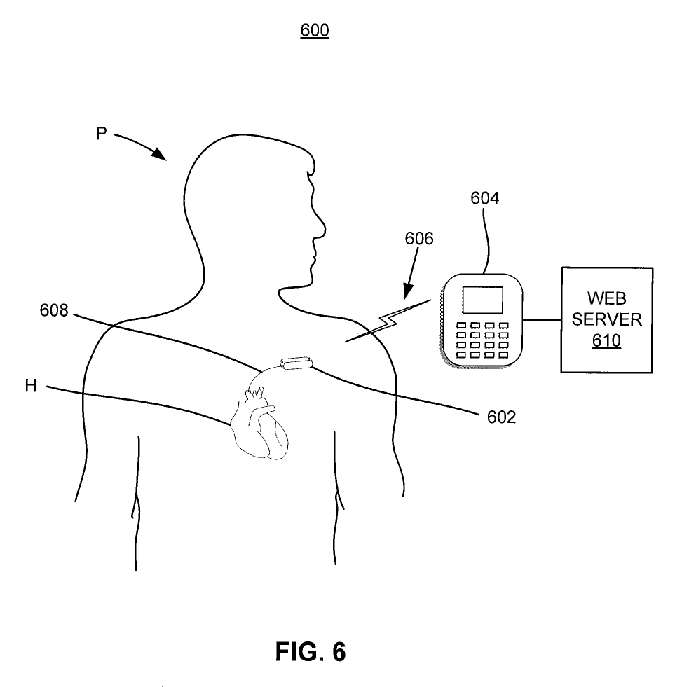

FIG. 6 illustrates a simplified diagram of a device 602 (implanted within a patient P) that communicates with a device 604 that is located external to the patient P. The implanted device 602 and the external device 604 may communicate with one another via a wireless communication link 606 (as represented by the depicted wireless symbol).

In the illustrated example, the implanted device 602 is an implantable cardiac device including one or more leads 608 that are routed to the heart H of the patient P. For example, the implanted device 602 may be a pacemaker, an implantable cardioverter defibrillator, or some other similar device. It should be appreciated, however, that the implanted device 602 may take other forms.

The external device 604 also may take various forms. For example, the external device 604 may be a base station, a programmer, a home safety monitor, a personal monitor, a follow-up monitor, a wearable monitor, or some other type of device that is configured to communicate with the implanted device 602.

The communication link 606 may be used to transfer information between the devices 602 and 604 in conjunction with various applications such as remote home-monitoring, clinical visits, data acquisition, remote follow-up, and portable or wearable patient monitoring/control systems. For example, when information needs to be transferred between the devices 602 and 604, the patient P moves into a position that is relatively close to the external device 604, or vice versa.

As mentioned above, an external device may send information it receives from an implanted device to another device (e.g., that may provide a more convenient means for a physician to review the information). For example, the external device 604 may send the information to a web server 610. In this way, the physician may remotely access the information (e.g., by accessing a website). The physician may then review the information uploaded from the implantable device to determine whether medical intervention is warranted.

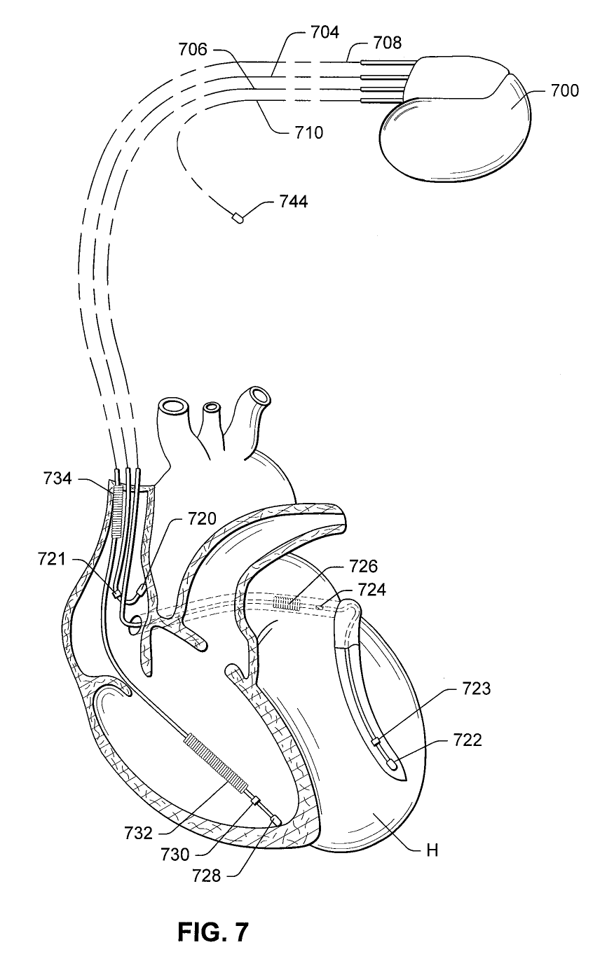

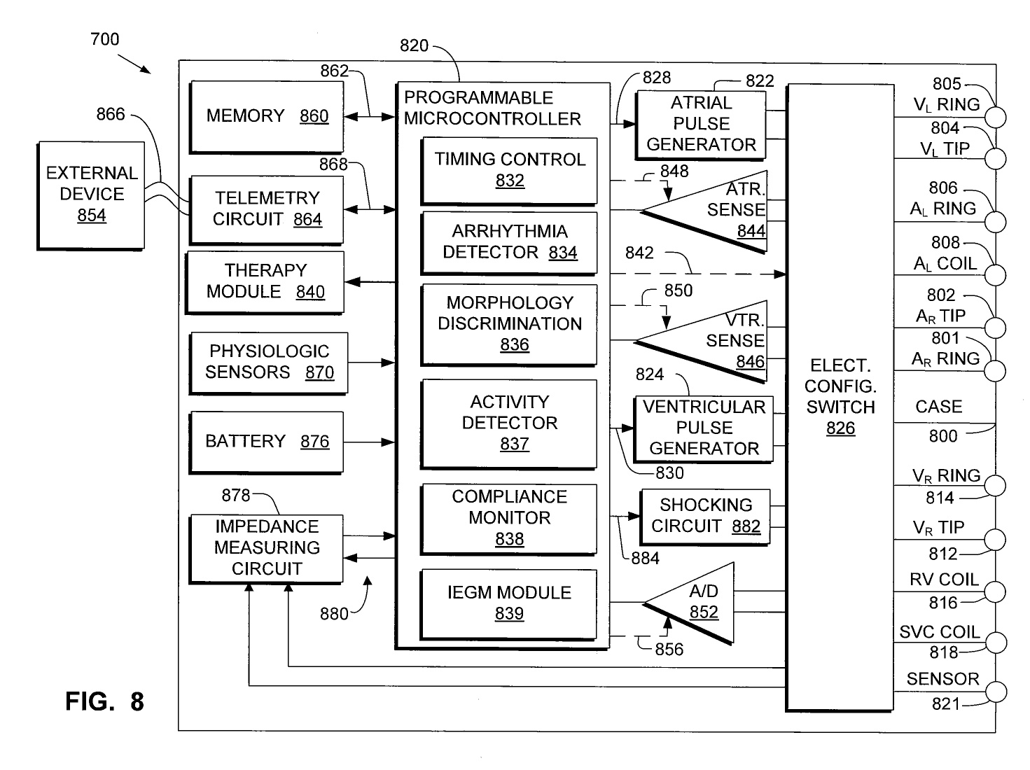

Referring now to FIGS. 7 and 8, an example of an implantable cardiac device (e.g., a stimulation device such as an implantable cardioverter defibrillator, a pacemaker, etc.) that may be implemented in accordance with the teachings herein will be described. It is to be appreciated and understood that other cardiac devices, including those that are not necessarily implantable, may be used and that the description below is given, in its specific context, to assist the reader in understanding, with more clarity, sample uses of the embodiments described herein.

FIG. 7 shows an exemplary implantable cardiac device 700 in electrical communication with a patient's heart H by way of three leads 704, 706, and 708, suitable for delivering multi-chamber stimulation and shock therapy. Bodies of the leads 704, 706, and 708 may be formed of silicone, polyurethane, plastic, or similar biocompatible materials to facilitate implant within a patient. Each lead includes one or more conductors, each of which may couple one or more electrodes incorporated into the lead to a connector on the proximal end of the lead. Each connector, in turn, is configured to couple with a complimentary connector (e.g., implemented within a header) of the device 700.

To sense atrial cardiac signals and to provide right atrial chamber stimulation therapy, the device 700 is coupled to an implantable right atrial lead 704 having, for example, an atrial tip electrode 720, which typically is implanted in the patient's right atrial appendage or septum. FIG. 7 also shows the right atrial lead 704 as having an optional atrial ring electrode 721.

To sense left atrial and ventricular cardiac signals and to provide left chamber pacing therapy, the device 700 is coupled to a coronary sinus lead 706 designed for placement in the coronary sinus region via the coronary sinus for positioning one or more electrodes adjacent to the left ventricle, one or more electrodes adjacent to the left atrium, or both. As used herein, the phrase “coronary sinus region” refers to the vasculature of the left ventricle, including any portion of the coronary sinus, the great cardiac vein, the left marginal vein, the left posterior ventricular vein, the middle cardiac vein, the small cardiac vein or any other cardiac vein accessible by the coronary sinus.

Accordingly, an exemplary coronary sinus lead 706 is designed to receive atrial and ventricular cardiac signals and to deliver left ventricular pacing therapy using, for example, a left ventricular tip electrode 722 and, optionally, a left ventricular ring electrode 723; provide left atrial pacing therapy using, for example, a left atrial ring electrode 724; and provide shocking therapy using, for example, a left atrial coil electrode 726 (or other electrode capable of delivering a shock). For a more detailed description of a coronary sinus lead, the reader is directed to U.S. Pat. No. 5,466,254, “Coronary Sinus Lead with Atrial Sensing Capability” (Helland), which is incorporated herein by reference.

The device 700 is also shown in electrical communication with the patient's heart H by way of an implantable right ventricular lead 708 having, in this implementation, a right ventricular tip electrode 728, a right ventricular ring electrode 730, a right ventricular (RV) coil electrode 732 (or other electrode capable of delivering a shock), and a superior vena cava (SVC) coil electrode 734 (or other electrode capable of delivering a shock). Typically, the right ventricular lead 708 is transvenously inserted into the heart H to place the right ventricular tip electrode 728 in the right ventricular apex so that the RV coil electrode 732 will be positioned in the right ventricle and the SVC coil electrode 734 will be positioned in the superior vena cava. Accordingly, the right ventricular lead 708 is capable of sensing or receiving cardiac signals, and delivering stimulation in the form of pacing and shock therapy to the right ventricle.

The device 700 is also shown in electrical communication with a lead 710 including one or more components 744 such as a physiologic sensor (e.g., a PPG sensor). The component 744 may be positioned in, near or remote from the heart.

It should be appreciated that the device 700 may connect to leads other than those specifically shown. In addition, the leads connected to the device 700 may include components other than those specifically shown. For example, a lead may include other types of electrodes, sensors or devices that serve to otherwise interact with a patient or the surroundings.

FIG. 8 depicts an exemplary, simplified block diagram illustrating sample components of the device 700. The device 700 may be adapted to treat both fast and slow arrhythmias with stimulation therapy, including cardioversion, defibrillation, and pacing stimulation. While a particular multi-chamber device is shown, it is to be appreciated and understood that this is done for illustration purposes. Thus, the techniques and methods described below can be implemented in connection with any suitably configured or configurable device. Accordingly, one of skill in the art could readily duplicate, eliminate, or disable the appropriate circuitry in any desired combination to provide a device capable of treating the appropriate chamber(s) with, for example, cardioversion, defibrillation, and pacing stimulation.

A housing 800 for the device 700 is often referred to as the “can”, “case” or “case electrode”, and may be programmably selected to act as the return electrode for all “unipolar” modes. The housing 800 may further be used as a return electrode alone or in combination with one or more of the coil electrodes 726, 732 and 734 for shocking purposes. The housing 800 may be constructed of a biocompatible material (e.g., titanium) to facilitate implant within a patient.

The housing 800 further includes a connector (not shown) having a plurality of terminals 801, 802, 804, 805, 806, 808, 812, 814, 816 and 818 (shown schematically and, for convenience, the names of the electrodes to which they are connected are shown next to the terminals). The connector may be configured to include various other terminals (e.g., terminal 821 coupled to a PPG sensor or some other component) depending on the requirements of a given application.

To achieve right atrial sensing and pacing, the connector includes, for example, a right atrial tip terminal (AR TIP) 802 adapted for connection to the right atrial tip electrode 720. A right atrial ring terminal (AR RING) 801 may also be included and adapted for connection to the right atrial ring electrode 721. To achieve left chamber sensing, pacing, and shocking, the connector includes, for example, a left ventricular tip terminal (VL TIP) 804, a left ventricular ring terminal (VL RING) 805, a left atrial ring terminal (AL RING) 806, and a left atrial shocking terminal (AL COIL) 808, which are adapted for connection to the left ventricular tip electrode 722, the left ventricular ring electrode 723, the left atrial ring electrode 724, and the left atrial coil electrode 726, respectively.

To support right chamber sensing, pacing, and shocking, the connector further includes a right ventricular tip terminal (VR TIP) 812, a right ventricular ring terminal (VR RING) 814, a right ventricular shocking terminal (RV COIL) 816, and a superior vena cava shocking terminal (SVC COIL) 818, which are adapted for connection to the right ventricular tip electrode 728, the right ventricular ring electrode 730, the RV coil electrode 732, and the SVC coil electrode 734, respectively.

At the core of the device 700 is a programmable microcontroller 820 that controls the various modes of stimulation therapy. As is well known in the art, microcontroller 820 typically includes a microprocessor, or equivalent control circuitry, designed specifically for controlling the delivery of stimulation therapy, and may further include memory such as RAM, ROM and flash memory, logic and timing circuitry, state machine circuitry, and I/O circuitry. Typically, microcontroller 820 includes the ability to process or monitor input signals (data or information) as controlled by a program code stored in a designated block of memory. The type of microcontroller is not critical to the described implementations. Rather, any suitable microcontroller 820 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander et al.) and U.S. Pat. No. 4,944,298 (Sholder), all of which are incorporated by reference herein. For a more detailed description of the various timing intervals that may be used within the device and their inter-relationship, see U.S. Pat. No. 4,788,980 (Mann et al.), also incorporated herein by reference.

FIG. 8 also shows an atrial pulse generator 822 and a ventricular pulse generator 824 that generate pacing stimulation pulses for delivery by the right atrial lead 704, the coronary sinus lead 706, the right ventricular lead 708, or some combination of these leads via an electrode configuration switch 826. It is understood that in order to provide stimulation therapy in each of the four chambers of the heart, the atrial and ventricular pulse generators 822 and 824 may include dedicated, independent pulse generators, multiplexed pulse generators, or shared pulse generators. The pulse generators 822 and 824 are controlled by the microcontroller 820 via appropriate control signals 828 and 830, respectively, to trigger or inhibit the stimulation pulses.

Microcontroller 820 further includes timing control circuitry 832 to control the timing of the stimulation pulses (e.g., pacing rate, atrio-ventricular (A-V) delay, atrial interconduction (A-A) delay, or ventricular interconduction (V-V) delay, etc.) or other operations, as well as to keep track of the timing of refractory periods, blanking intervals, noise detection windows, evoked response windows, alert intervals, marker channel timing, etc., as known in the art.

Microcontroller 820 further includes an arrhythmia detector 834. The arrhythmia detector 834 may be utilized by the device 700 for determining desirable times to administer various therapies. The arrhythmia detector 834 may be implemented, for example, in hardware as part of the microcontroller 820, or as software/firmware instructions programmed into the device 700 and executed on the microcontroller 820 during certain modes of operation.

Microcontroller 820 may include a morphology discrimination module 836, a capture detection module (not shown), and an auto sensing module (not shown). These modules are optionally used to implement various exemplary recognition algorithms or methods. The aforementioned components may be implemented, for example, in hardware as part of the microcontroller 820, or as software/firmware instructions programmed into the device 700 and executed on the microcontroller 820 during certain modes of operation.

The electrode configuration switch 826 includes a plurality of switches for connecting the desired terminals (e.g., that are connected to electrodes, coils, sensors, etc.) to the appropriate I/O circuits, thereby providing complete terminal and, hence, electrode programmability. Accordingly, switch 826, in response to a control signal 842 from the microcontroller 820, may be used to determine the polarity of the stimulation pulses (e.g., unipolar, bipolar, combipolar, etc.) by selectively closing the appropriate combination of switches (not shown) as is known in the art.

Atrial sensing circuits (ATR. SENSE) 844 and ventricular sensing circuits (VTR. SENSE) 846 may also be selectively coupled to the right atrial lead 704, coronary sinus lead 706, and the right ventricular lead 708, through the switch 826 for detecting the presence of cardiac activity in each of the four chambers of the heart. Accordingly, the atrial and ventricular sensing circuits 844 and 846 may include dedicated sense amplifiers, multiplexed amplifiers, or shared amplifiers. Switch 826 determines the “sensing polarity” of the cardiac signal by selectively closing the appropriate switches, as is also known in the art. In this way, the clinician may program the sensing polarity independent of the stimulation polarity. The sensing circuits (e.g., circuits 844 and 846) are optionally capable of obtaining information indicative of tissue capture.

Each sensing circuit 844 and 846 preferably employs one or more low power, precision amplifiers with programmable gain, automatic gain control, bandpass filtering, a threshold detection circuit, or some combination of these components, to selectively sense the cardiac signal of interest. The automatic gain control enables the device 700 to deal effectively with the difficult problem of sensing the low amplitude signal characteristics of atrial or ventricular fibrillation.

The outputs of the atrial and ventricular sensing circuits 844 and 846 are connected to the microcontroller 820, which, in turn, is able to trigger or inhibit the atrial and ventricular pulse generators 822 and 824, respectively, in a demand fashion in response to the absence or presence of cardiac activity in the appropriate chambers of the heart. Furthermore, as described herein, the microcontroller 820 is also capable of analyzing information output from the sensing circuits 844 and 846, a data acquisition system 852, or both. This information may be used to determine or detect whether and to what degree tissue capture has occurred and to program a pulse, or pulses, in response to such determinations. The sensing circuits 844 and 846, in turn, receive control signals over signal lines 848 and 850, respectively, from the microcontroller 820 for purposes of controlling the gain, threshold, polarization charge removal circuitry (not shown), and the timing of any blocking circuitry (not shown) coupled to the inputs of the sensing circuits 844 and 846 as is known in the art.

For arrhythmia detection, the device 700 utilizes the atrial and ventricular sensing circuits 844 and 846 to sense cardiac signals to determine whether a rhythm is physiologic or pathologic. It should be appreciated that other components may be used to detect arrhythmia depending on the system objectives. In reference to arrhythmias, as used herein, “sensing” is reserved for the noting of an electrical signal or obtaining data (information), and “detection” is the processing (analysis) of these sensed signals and noting the presence of an arrhythmia.

Timing intervals between sensed events (e.g., P-waves, R-waves, and depolarization signals associated with fibrillation) may be classified by the arrhythmia detector 834 of the microcontroller 820 by comparing them to a predefined rate zone limit (e.g., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules may be applied to the atrial channel to determine if there is an atrial tachyarrhythmia or atrial fibrillation with appropriate classification and intervention.

Cardiac signals or other signals may be applied to inputs of an analog-to-digital (A/D) data acquisition system 852. The data acquisition system 852 is configured (e.g., via signal line 856) to acquire intracardiac electrogram (“IEGM”) signals or other signals, convert the raw analog data into a digital signal, and store the digital signals for later processing, for telemetric transmission to an external device 854, or both. For example, the data acquisition system 852 may be coupled to the right atrial lead 704, the coronary sinus lead 706, the right ventricular lead 708 and other leads through the switch 826 to sample cardiac signals across any pair of desired electrodes.

The data acquisition system 852 also may be coupled to receive signals from other input devices. For example, the data acquisition system 852 may sample signals from a physiologic sensor 870 or other components shown in FIG. 8 (connections not shown).

The microcontroller 820 is further coupled to a memory 860 by a suitable data/address bus 862, wherein the programmable operating parameters used by the microcontroller 820 are stored and modified, as required, in order to customize the operation of the device 700 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart H within each respective tier of therapy. One feature of the described embodiments is the ability to sense and store a relatively large amount of data (e.g., from the data acquisition system 852), which data may then be used for subsequent analysis to guide the programming of the device 700.

Advantageously, the operating parameters of the implantable device 700 may be non-invasively programmed into the memory 860 through a telemetry circuit 864 in telemetric communication via communication link 866 with the external device 854, such as a programmer, transtelephonic transceiver, a diagnostic system analyzer or some other device. The microcontroller 820 activates the telemetry circuit 864 with a control signal (e.g., via bus 868). The telemetry circuit 864 advantageously allows intracardiac electrograms and status information relating to the operation of the device 700 (as contained in the microcontroller 820 or memory 860) to be sent to the external device 854 through an established communication link 866.

The device 700 can further include one or more physiologic sensors 870 (e.g., a PPG sensor, a pressure sensor, etc.). In some embodiments the device 700 may include a “rate-responsive” sensor that may provide, for example, information to aid in adjustment of pacing stimulation rate according to the exercise state of the patient. One or more physiologic sensors 870 may further be used to detect changes in cardiac output, changes in the physiological condition of the heart, or diurnal changes in activity (e.g., detecting sleep and wake states). Accordingly, the microcontroller 820 responds by adjusting the various pacing parameters (such as rate, A-V Delay, V-V Delay, etc.) at which the atrial and ventricular pulse generators 822 and 824 generate stimulation pulses.

While shown as being included within the device 700, it is to be understood that a physiologic sensor 870 may also be external to the device 700, yet still be implanted within or carried by the patient. Examples of physiologic sensors that may be implemented in conjunction with the device 700 include sensors that sense respiration rate, pH of blood, ventricular gradient, oxygen saturation, blood pressure and so forth. Another sensor that may be used is one that detects activity variance, wherein an activity sensor is monitored diurnally to detect the low variance in the measurement corresponding to the sleep state. For a more detailed description of an activity variance sensor, the reader is directed to U.S. Pat. No. 5,476,483 (Bornzin et al.), which patent is hereby incorporated by reference.

The one or more physiologic sensors 870 may optionally include one or more of components to help detect movement (via, e.g., a position sensor or an accelerometer) and minute ventilation (via an MV sensor) in the patient. Signals generated by the position sensor and MV sensor may be passed to the microcontroller 820 for analysis in determining whether to adjust the pacing rate, etc. The microcontroller 820 may thus monitor the signals for indications of the patient's position and activity status, such as whether the patient is climbing up stairs or descending down stairs or whether the patient is sitting up after lying down.

The device 700 additionally includes a battery 876 that provides operating power to all of the circuits shown in FIG. 8. For a device 700 which employs shocking therapy, the battery 876 is capable of operating at low current drains (e.g., preferably less than 10 μA) for long periods of time, and is capable of providing high-current pulses (for capacitor charging) when the patient requires a shock pulse (e.g., preferably, in excess of 2 A, at voltages above 200 V, for periods of 10 seconds or more). The battery 876 also desirably has a predictable discharge characteristic so that elective replacement time can be detected. Accordingly, the device 700 preferably employs lithium or other suitable battery technology.

The device 700 can further include magnet detection circuitry (not shown), coupled to the microcontroller 820, to detect when a magnet is placed over the device 700. A magnet may be used by a clinician to perform various test functions of the device 700 and to signal the microcontroller 820 that the external device 854 is in place to receive data from or transmit data to the microcontroller 820 through the telemetry circuit 864.

The device 700 further includes an impedance measuring circuit 878 that is enabled by the microcontroller 820 via a control signal 880. The known uses for an impedance measuring circuit 878 include, but are not limited to, lead impedance surveillance during the acute and chronic phases for proper performance, lead positioning or dislodgement; detecting operable electrodes and automatically switching to an operable pair if dislodgement occurs; measuring respiration or minute ventilation; measuring thoracic impedance for determining shock thresholds; detecting when the device 700 has been implanted; measuring stroke volume; and detecting the opening of heart valves, etc. The impedance measuring circuit 878 is advantageously coupled to the switch 826 so that any desired electrode may be used.

In the case where the device 700 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 820 further controls a shocking circuit 882 by way of a control signal 884. The shocking circuit 882 generates shocking pulses of low (e.g., up to 0.5 J), moderate (e.g., 0.5 J to 10 J), or high energy (e.g., 11 J to 40 J), as controlled by the microcontroller 820. Such shocking pulses are applied to the patient's heart H through, for example, two shocking electrodes and as shown in this embodiment, selected from the left atrial coil electrode 726, the RV coil electrode 732 and the SVC coil electrode 734. As noted above, the housing 800 may act as an active electrode in combination with the RV coil electrode 732, as part of a split electrical vector using the SVC coil electrode 734 or the left atrial coil electrode 726 (i.e., using the RV electrode as a common electrode), or in some other arrangement.

Cardioversion level shocks are generally considered to be of low to moderate energy level (so as to minimize pain felt by the patient), be synchronized with an R-wave, pertain to the treatment of tachycardia, or some combination of the above. Defibrillation shocks are generally of moderate to high energy level (i.e., corresponding to thresholds in the range of 5 J to 40 J), delivered asynchronously (since R-waves may be too disorganized), and pertaining to the treatment of fibrillation. Accordingly, the microcontroller 820 is capable of controlling the synchronous or asynchronous delivery of the shocking pulses.

As mentioned above, the device 700 may include several components that provide compliance monitoring functionality as taught herein. For example, one or more of the switch 826, the sense circuits 844, 846, and the data acquisition system 852 may acquire signals that are used in the compliance monitoring operations discussed above, with reference to FIGS. 1-6. The data described above may be stored in the memory 860. In addition, in some implementations a therapy module 840 (e.g., corresponding to therapy module 112) may be provided to facilitate the administration of therapy based on the monitoring.

The microcontroller 820 (e.g., a processor providing signal processing functionality) also may implement or support at least a portion of the compliance monitoring-related functionality discussed herein. For example, an activity detector 837 may perform activity detection as described above with reference to FIG. 6. A compliance monitor 838 may perform compliance monitoring operations as described above with reference to FIGS. 1-6. In addition, an IEGM module 839 may be used to acquire IEGM data that may be used by the above components (e.g., to determine timing of cardiac events such as R-waves).

It should be appreciated from the above that the various structures and functions described herein may be incorporated into a variety of apparatuses (e.g., a stimulation device, a lead, a monitoring device, etc.) and implemented in a variety of ways. Different embodiments of such an apparatus may include a variety of hardware and software processing components. In some embodiments, hardware components such as processors, controllers, state machines, logic, or some combination of these components, may be used to implement the described components or circuits.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

Moreover, some of the operations described herein may be performed by a device that is located externally with respect to the body of the patient. For example, an implanted device may send raw data or processed data to an external device that then performs the necessary processing.

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, light pulses transmitted through an optical medium such as an optical fiber or air, or RF waves transmitted through a medium such as air, and so on. In addition, a plurality of signals may be collectively referred to as a signal herein. The signals discussed above also may take the form of data. For example, in some embodiments an application program may send a signal to another application program. Such a signal may be stored in a data memory.

Moreover, the recited order of the blocks in the processes disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above it will be understood that the teachings herein are intended to cover any changes, adaptations or modifications which are within the scope of the disclosure.

What is claimed is:

1. An implantable medical monitoring apparatus, comprising:
a reflectance time detector configured to determine reflectance time of a pressure wave in a cardiovascular system of a patient;
a blood pressure detector configured to determine blood pressure of the cardiovascular system; and
a cardiovascular vessel elasticity detector configured to determine elasticity of a vessel of the cardiovascular system based on the determined blood pressure, the determined reflectance time, and a data set that correlates different blood pressure values and different pressure wave reflectance time values to different vessel elasticity values.

2. The apparatus of claim 1, wherein the data set comprises a set of iso-pressure lines that map reflectance time to elasticity.

3. The apparatus of claim 1, further comprising a photoplethysmography sensor configured to generate signals indicative of changes in blood flow in the cardiovascular system, wherein the reflectance time detector is further configured to process the generated signals to determine the reflectance time.

4. The apparatus of claim 3, wherein the blood pressure detector is further configured to process the generated signals to determine the blood pressure.

5. The apparatus of claim 1, wherein the cardiovascular vessel elasticity detector is further configured to determine a trend indicative of changes in elasticity of the vessel based on the determined elasticity.

6. The apparatus of claim 5, further comprising a therapy module configured to identify at least one condition associated with the patient based on the trend.

7. The apparatus of claim 6, wherein the at least one condition relates to at least one of the group consisting of: an indication of heart failure, an indication of hypertension, an indication of neurohormonal insult leading to wall stiffness, medical regimen compliance, and salt intake compliance.

8. The apparatus of claim 1, wherein:
the data set comprises a set of iso-pressure lines; and
the cardiovascular vessel elasticity detector is further configured to determine a trend indicative of changes in elasticity of the vessel based on the determined elasticity.

9. The apparatus of claim 8, further comprising a photoplethysmography sensor configured to generate signals indicative of changes in blood flow in the cardiovascular system, wherein:
the reflectance time detector is further configured to process the generated signals to determine the reflectance time; and
the blood pressure detector is further configured to process the generated signals to determine the blood pressure.

10. A method of monitoring vessel elasticity, comprising:
determining reflectance time of a pressure wave in a cardiovascular system of a patient;
determining blood pressure of the cardiovascular system; and
determining elasticity of a vessel of the cardiovascular system based on the determined blood pressure, the determined reflectance time, and a data set that correlates different blood pressure values and different pressure wave reflectance time values to different vessel elasticity values.

11. The method of claim 10, wherein the data set comprises a set of iso-pressure lines that map reflectance time to elasticity.

12. The method of claim 10, wherein the determination of the reflectance time comprises using photoplethysmography to detect the pressure wave.

13. The method of claim 12, wherein the determination of the blood pressure comprises using photoplethysmography to detect blood velocity in the cardiovascular system.

14. The method of claim 10, further comprising:
determining a trend indicative of changes in elasticity of the vessel based on the determined elasticity; and
identifying at least one condition associated with the patient based on the trend.

15. The method of claim 14, wherein the at least one condition relates to at least one of the group consisting of: an indication of heart failure, an indication of hypertension, an indication of neurohormonal insult leading to wall stiffness, medical regimen compliance, and salt intake compliance.

16. The method of claim 10, wherein the data set comprises a set of iso-pressure lines, the method further comprising:
determining a trend indicative of changes in elasticity of the vessel based on the determined elasticity; and
predicting heart failure or hypertension based on the trend.

17. The method of claim 16, wherein:
the determination of the reflectance time comprises using photoplethysmography to measure changes in blood flow associated with the pressure wave; and
the determination of the blood pressure comprises using photoplethysmography to measure changes in blood flow in the cardiovascular system and estimating the blood pressure based on the changes in blood flow in the cardiovascular system.

18. The method of claim 10, wherein the blood pressure and the reflectance time are determined by an implantable medical device.
